(19) United States
(12) Patent Application Publication
(10) Pub. No.: US 2004/0116360 A1
(43) Pub. Date: Jun. 17, 2004

(54) Encapsulation and deaggregation of polyene antibiotics using poly(ethylene glycol)-phospholipid micelles

(76) Inventor: Glen S. Kwon, Waunakee, WI (US)

Correspondence Address: GREENLEE WINNER AND SULLIVAN P C, 5370 MANHATTAN CIRCLE, SUITE 201, BOULDER, CO, 80303, US

(21) Appl. No.: 10/687,558

(22) Filed: Oct. 15, 2003

Related U.S. Application Data
(60) Provisional application No. 60/418,927, filed on Oct. 15, 2002.

(51) Int. Cl. A61K 31/7048; A61K 9/127

(52) U.S. Cl. 514/28; 424/450

(57) ABSTRACT

AmphotericinB (or other hydrophobic compound) is encapsulated in a deaggregated form in micelles of monomethoxy poly(ethylene glycol)-phospholipid (as specifically exemplified, the phospholipid is 1,2 di-stearoyl-sn-glycero-3-phosphatidyl ethanolamine) formed by solvent evaporation. Advantageously, the hydration of the dried drug-polymer film is carried at between about 25° C., and about 80° C. The micelles can be reconstituted with the Amphotericin B (or other hydrophobic compound) in a deaggregated state and safely used in therapy for fungal infections of humans or animals, especially for systemic fungal infections, or other desired application. The polyene micellar formulations described herein are reduced in toxicity as compared with those polyene formulations in which there is significant occurrence of aggregated polyenes.

### CROSS REFERENCE TO RELATED APPLICATIONS

This application claims benefit of U.S. Provisional Application No. 60/418,927, filed Oct. 15, 2002.

### ACKNOWLEDGMENT OF FEDERAL RESEARCH SUPPORT

This invention was made, at least in part, with funding from the National Institutes of Health (Grant No. A143346). Accordingly, the United States Government has certain rights in this invention.

### BACKGROUND OF THE INVENTION

The field of the present invention is the area of methods of formulating pharmaceutical compositions for medical and/or veterinary use, in particular, methods of formulating relatively insoluble or toxic materials such as polyene antibiotics, e.g., amphotericin B and nystatin; anticancer drugs, e.g., paclitaxel and comptothecin, hydrophobic prodrugs; and the like, so that solubility in aqueous milieus is improved and so that toxicity is reduced, release is controlled and in at least some instances, the stability of the formulation is improved.

Fungal infections are, in part, associated with immune-compromised patients such as those infected with HIV, patients who have been subjected to anticancer therapeutics or immune suppressive drugs after organ transplants, and the elderly. Fungal infections fall into two categories: systemic (deep) mycoses and superficial mycoses that involve the skin or mucous membranes. The dermatophytic fungi infect the skin, hair and nails; etiological agents include Epidermiphyton spp., Trichophyton spp. and Microspermum spp. Generally, infections of the mucous membranes are due to infections with Candida albicans. The systemic mycoses are serious and often life-threatening. They include cryptococcosis, systemic candidiasis, aspergillosis, blastomycosis, histoplasmosis, coccidiodomycosis, paracoccidioidomycosis, phycomycosis, torulopsosis, among others.

The three families of drugs used to treat fungal infections are the polyenes, imidazoles and antimetabolites. The polyenes include nystatin, which is generally used for superficial infections only, and amphotericin B. Mepartricin and natrimycin are other polyenes with antifungal activities.

Ketoconazole, miconazole and thiabendazole are imidazoles with antifungal activity. They act by inhibiting cytochrome activity and by interfering with ergosterol synthesis. Flucytosine is an antimetabolite which has been used in the treatment of systemic mycoses. It is converted in vivo to 5-fluorouracil, which inhibits thymidylate synthetase.

Amphotericin B (AmB) has an affinity for membranes with a relatively high ergosterol content; it forms channels which allow the passage of potassium and other small molecules. Because the AmB is very toxic, especially in aggregated form, and it has numerous side effects, it must be given in a hospital setting, adding to treatment costs. There is some evidence (Beringue et al. (1999) J. Gen. Virol. 80, 1873-1877; Beringue et al. (2000) J. Virol. 74, 5432-5440) that certain polyenes may inhibit the progression of scrapie infections.

Despite its low solubility in water and the toxicity problems, AmB is one of the drugs of choice for treating fungal infections. Notably, the development of resistance to AmB is very rare. Numerous strategies have been employed to improve its solubility in aqueous systems and to reduce its toxicity. Strategies for the improvement of solubility and toxicity have included formulation with surfactant, e.g. deoxycholate, liposome encapsulation, encapsulation in polyethylene glycol-complexed liposomes and encapsulation with various amphiphilic polymeric materials.

Detergents such as sodium deoxycholate have been used to solubilize and/or deaggregate AmB. While the deaggregation provides a reduction in the toxicity of the AmB, the solubilizing agent itself is toxic if the levels administered to a patient are sufficiently high (J. Barwicz et al. [1992] Antimicrob. Agents Chemother. 36:2310-2315). Excess sodium deoxycholate or excess sodium lauryl sulfate (50:1 ratio of surfactant to AmB) is toxic. However, Tween 80 (trademark of Uniqema; polyoxyethylenesorbitan monooleate) did not appear to deaggregate AmB. In addition, U.S. Pat. No. 6,013,283 (Greenwald et al., 2000) does not appear to teach deaggregation of AmB by mPEG-DSPE.

Polyoxyethylene glycol(24) cholesterol has been complexed with AmB to reduce toxicity as measured by hemolysis (Tasset et al. 1990, Internat. J. Pharmaceutics 58:41-48). However, the polymer itself has significant hemolytic activity, as shown in FIG. 1 of this reference.

Because both cancer and fungal infections are relatively difficult to treat, because cancer and systemic fungal infections are often life-threatening, and because the antifungal antibiotics as well as most cancer chemotherapeutic agents are often toxic to animals, including humans, there is a long-felt need in the art for pharmaceutical compositions comprising polyene antibiotics and other relatively toxic, hydrophobic therapeutic agents, which compositions are improved in relative toxicity to the patient and in release properties. There is also need in the art to formulate other hydrophobic molecules so that water solubility is improved.

### SUMMARY OF THE INVENTION

The present invention provides improved methods and compositions for the formulation of amphotericin B in a form characterized in that there is less aggregation of the AmB than in prior art formulations, and therefore the compositions of the present invention are less toxic than certain other formulations of AmB. Methoxy poly(ethylene glycol)-phospholipid (mPEG-PL) AmB micelles at relatively low mPEG-PL:AmB ratios deaggregate AmB and thereby reduce its toxicity without a concurrent loss of antifungal activity. As specifically exemplified, the phospholipid is 1,2 di-stearoyl-sn-glycero-3-phosphatidylethanolamine (DSPE). Desirably, the molecular weight of the mPEG-DSPE is between about 1500 and about 12,000, preferably 2800-6500. Other phospholipid components can include the lauryl, myristoyl, palmitoyl, oleoyl and linoleoyl analogs of the stearoyl-substituted phosphatidyl ethanolamine polymer. Preferred mPEG-DSPE:AmB molar ratios are from about 0:75:1 to about 3:1, and desirably the ratio is about 1:1 to about 1.5:1. These methods can be applied to other polyene antibiotics including, but not limited to, nystatin, and to unrelated hydrophobic therapeutic agents such as paclitaxel or comptothecin, and prodrugs, as well as to hydrophobic compounds other than pharmaceuticals. While monomethoxy PEG-PL was used the experiments described herein, it is understood that other monoalkoxy PEG derivatives could be used in PEG-PL polymers in the methods of the present invention. Functionalized PEG, as known to the art, can also be incorporated in the PEG-PL polymers for micellization as described herein.

The micelles of the present invention are prepared by dissolving the mPEG-PL and the passenger compound in a solvent, removing the solvent by evaporation under conditions of reduced pressure and elevated temperature to produce a thin film comprising mPEG-PL and passenger compound, and adding water to the thin film, preferably at a temperature from room temperature (about 25° C.) to about 80° C., preferably a temperature at or above the phase (glass) transition temperature of the PL but below the temperature at which the passenger compound decomposes or loses activity, to produce micelles. In general, increasing the temperature above room temperature improves micellization and/or loading of the passenger compound into the micelles. For micelles comprising AmB and mPEG-DSPE, the drug and polymer are dissolved in methanol or chloroform:methanol (1:2), but other solvents can be used. Hydration of the thin film to form micelles is carried out from about 20° C. to about 80° C., desirably at about 40° C. to about 75° C. or from about 55 to about 75° C. Paclitaxel has also been incorporated into micelles with mPEG-DSPE.

### BRIEF DESCRIPTION OF THE DRAWINGS

FIG. 1A shows the structure of mPEG-DSPE (MW 5770 g/mole). FIG. 1B is a schematic of a mPEG-DSPE conjugate micelle. The critical micelle concentration is 10 μg/ml, as determined in pyrene using a fluorescent probe.

FIG. 2 shows that mPEG-DSPE micelles encapsulate AmB after a solvent evaporation method of drug loading.

FIG. 3 shows spectra (300 to 450 nm) for solutions containing different concentrations of AmB-containing micelles of mPEG-DSPE, prepared by solvent evaporation (in distilled water), determined according to Example 2 herein below.

FIG. 4 shows the effect of molar ratio of mPEG-DSPE/AmB on the aggregation state of AmB in water.

FIG. 5A shows the relationship between dosage of mPEG-DSPE/AmB in micelles (administered intravenously) and time in a neutropenic mouse model of disseminated candidiasis.

FIG. 5B shows the effect of AmB-deoxycholate in a neutropenic mouse model of disseminated candidiasis.

FIG. 6 is a standard curve showing the linear relationship between absorbance at 412 nm and concentration of AmB (micrograms/milliliter) in DMF:water (1:1).

FIG. 7A illustrates the linear relationship between the theoretical and experimental ratios of polymer:AmB.

FIG. 7B shows the relationship of percentage yield and theoretical molar ratio of polymer to drug (mPEG-DSPE/AmB).

FIG. 7C illustrates the relationship of the PeakI/Peak IV absorbance and the theoretical molar ratio of polymer to drug (mPEG-DSPE/AmB).

FIG. 8A provides a contour plot of the Peak I/Peak IV absorbance as a function of mPEG-DSPE concentration (micrograms per 0.5 milliliter).

FIG. 8B illustrates the relationship of the theoretical concentration of mPEG-DSPE and theoretical concentration of AmB (micrograms per 0.5 milliliter).

FIG. 9 shows the emission spectra of mPEG-DSPE concentrate in 0.6 μM pyrene.

FIG. 10 compares the hemolytic activity against mouse red blood cells of Amphotericin B dissolved in DMSO with Amphotericin B in mPEG-DSPE micelles. (2k) refers to an mPEG component of 2000 d; (5k) refers to an mPEG component of 5000 d.

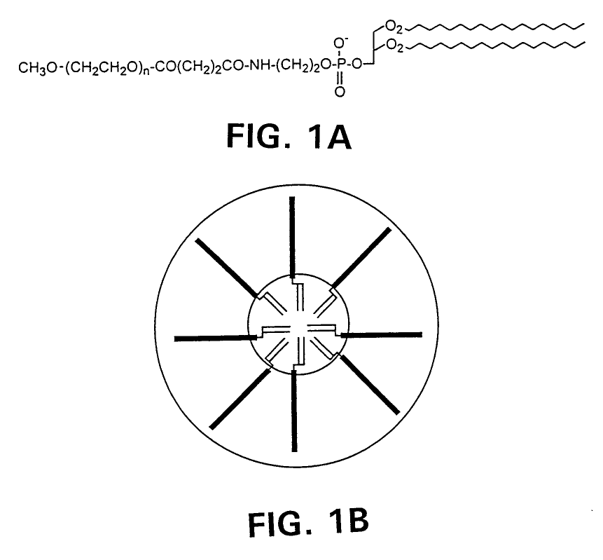

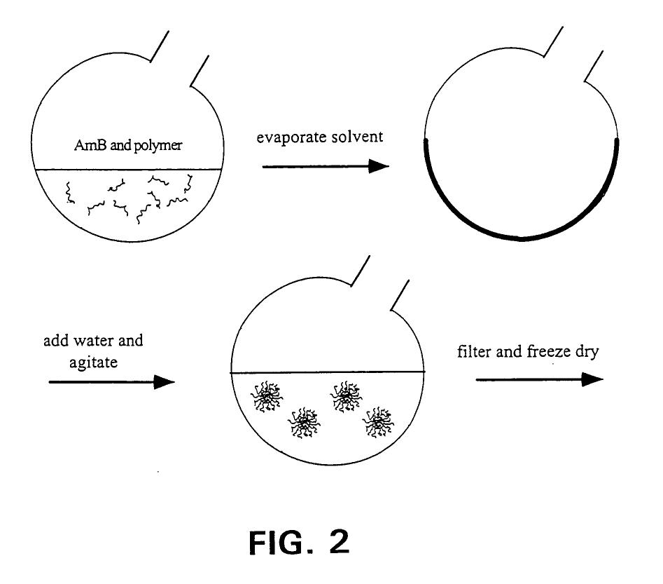

### DETAILED DESCRIPTION OF THE INVENTION

Abbreviations used in the present disclosure include the following: mPEG-PL monomethoxy poly(ethylene glycol)-phospholipid; mPEG-DSPE, monomethoxy (polyethylene glycol)-1,2-di-stearoyl-phosphatidyl ethanolamine (mPEG-DSPE), DSPE-PEG, Distearoyl-N-(monomethoxy poly(ethylene glycol) succinyl phosphatidyl ethanolamine; mPEG-b-PLAA, monomethoxy poly(ethylene glycol)-block-poly(L-aspartic acid); mPEG-b-PHSA, monomethoxy poly(ethylene glycol)-block-poly(N-hexyl stearate L-aspartamide); mPEG-b-PBLA, monomethoxy poly(ethylene glycol)-block-poly(P-benzyl-L-aspartate); mPEG-b-PHCA, monomethoxy poly(ethylene glycol)-block-poly(N-hexyl caprate L-aspartamide) mPEG-b-PHHA, monomethoxy poly(ethylene oxide)-block-poly(hydroxyhexyl L-aspartamide); AmB, Amphotericin B; DMSO, N,N′-dimethylsulfoxide; DMF, N,N′-dimethylformamide; SEC, Size exclusion chromatography; RBC, red blood cell; PBS, phosphate buffered saline; MIC, minimum inhibitory concentration; CFU, colony forming units; iv, intravenous.

The solvent evaporation method used to encapsulate AmB in mPEG-b-PHSA micelles is used to prepare the micelles of the present invention. AmB and mPEG-DSPE were dissolved in methanol, and a thin film of polymer and drug was coated on a round bottom flask by evaporation of methanol under vacuum with heat. Distilled water was added to dissolve the film and form mPEG-DSPE micelles with encapsulated AmB, and the micellar solution was filtered (0.22 μm) and freeze-dried.

AmB has two distinct electronic absorption spectra according to its molecular conformation. After AmB is dissolved in an organic solvent such as DMSO or DMF and diluted with water, AmB has a spectrum characterized by a broad band at 328 nm (A form). Such a spectrum corresponds to the highly aggregated species of AmB. The critical aggregation concentration of AmB in water is about 1.0 μg/ml. By contrast, AmB is entirely monomeric in DMF or DMSO, and in such solvents it has a characteristic spectrum with four well-separated bands at 350, 368, 388 and 412 nm (B form) (Rinnert et al. (1997) Biopolymers 16: 2419-2427).

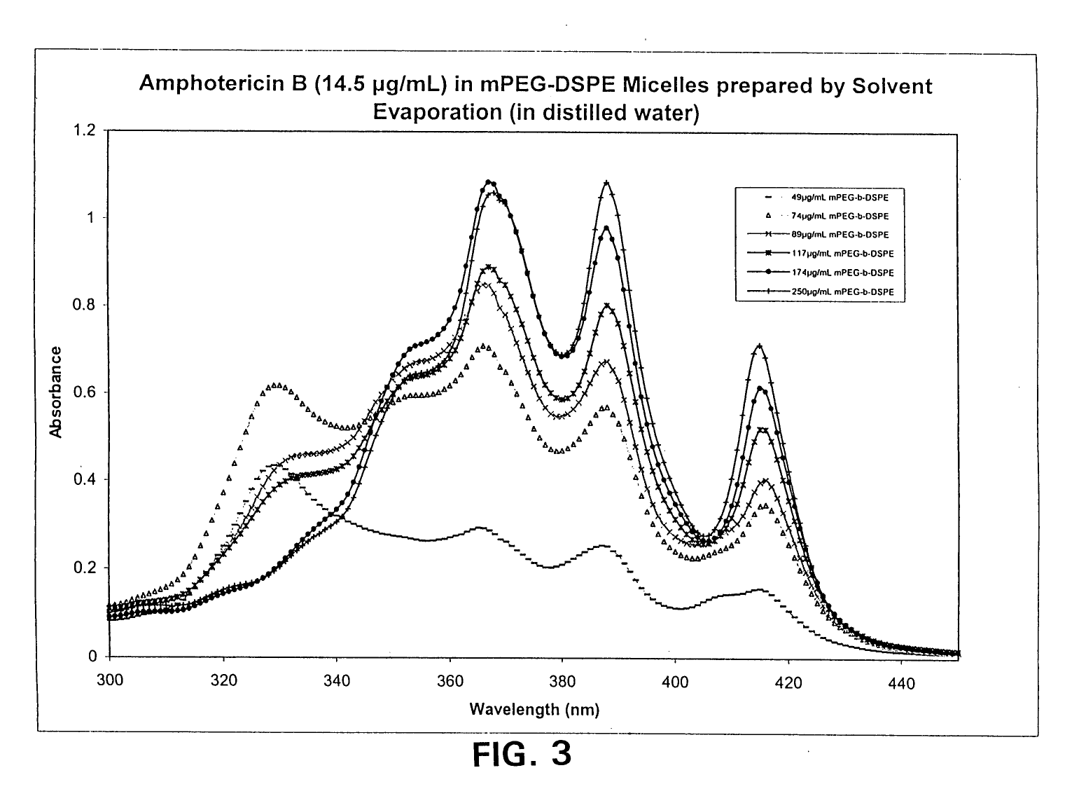

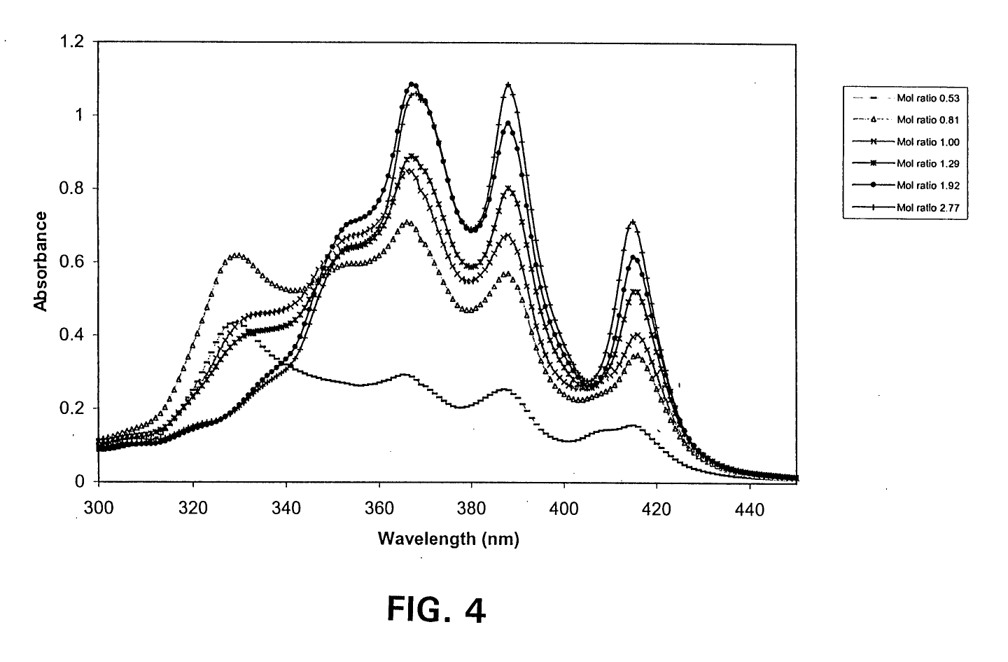

At a relatively low mPEG-DSPE:AmB ratio, AmB at a level of 14.5 μg/ml has a spectrum indicating aggregated drug. See FIG. 3. However, as the mPEG-DSPE:AmB ratio increases, there is a noticeable change in the absorption spectrum of AmB. There is a loss of absorbance at 328 nm and an increase in the absorbance of bands associated with monomeric drug. Therefore, mPEG-DSPE micelles encapsulate AmB in a deaggregated state at an appropriate ratio of mPEG-DSPE:AmB. As encapsulated in the micellar preparations of the present invention, AmB interacts with stearoyl chains in the cores of micelles instead of self-aggregating. Deaggregation occurs at fairly low mPEG-DSPE:AmB ratio (<4). In contrast, surfactants such as sodium deoxycholate require molar ratios greater than 40 to efficiently deaggregate AmB, and there is the risk of toxicity in a patient due to the surfactant as well as the therapeutic agent. Polyoxamers are unable to deaggregate AmB after solvent evaporation [Forster et al. (1988) J. Pharm. Pharmacol. 30: 325-328). AmB encapsulated in mPEG-DSPE micelles can reach levels of greater than 240 μg/ml after reconstitution.

The absorption spectra of several preparations of AmB-mPEG-DSPE micelles resuspended in water were determined. The results are shown in FIG. 3. The ratios calculated for absorbances at 328 nm and at 412 nm indicated ratios of AmB:polymer from 0.53 to 2.77. The results show that where the molar ratio of mPEG-DSPE:AmB is greater than about 1:1, and up to about 10:1, the AmB in the micellar preparation in water is primarily in the deaggregated form, and the toxicity is predicted to be low.

Liposomes containing mPEG-DSPE have a long half-life in circulation in humans and other animals. Such liposomes have been safely used in humans [Gregoriadis, G. (1995) TIBTECH 13:527-537]. The use of mPEG-DSPE micelles for the formulation and delivery of drugs (other than AmB) which are poorly soluble in water has been reviewed [Torchilin, V. P. (2001) J. Controlled Release 73: 137-172].

The mPEG-PL-AmB micelles of the present invention, prepared by solvent evaporation and reconstitution, can be used to treat systemic fungal infections safely and efficaciously due to the deaggregated state of the AmB in these micelles. These encapsulated AmB-containing compositions of the present invention are improved with respect to the deaggregated state of AmB, and therefore, with respect to toxicity and with respect to release properties. The present compositions are effective in inhibiting the growth of representative fungal pathogens in vitro. These compositions are similarly effective in vivo after administration by a parenteral route, desirably by intravenous injection, and especially by intravenous perfusion, for the treatment of systemic fungal infections. Therapeutic dosages can range from about 0.1 to about 5 mg/kg/day. Alternatively, the compositions of the present invention can be formulated for use as topical therapeutics for fungal infections of the skin, fingernails, toenails or hair, or for fungal infections of mucosal surfaces (oral or vaginal). The material used to complex with the AmB is a di-substituted fatty acyl derivative of mPEG (lauryl, myristoyl, palmitoyl, oleoyl, linoleoyl, stearoyl, desirably stearoyl). Di-substituted fatty acyl derivatives of mPEG are required in dramatically lower amounts (ratios) with AmB to achieve deaggregation of AmB and low toxicity.

Pathogenic fungi against which the AmB of the present invention are effective include, without limitation, species of Histoplasma, Cryptococcus, Candida, Aspergillus, Blastomyces, Mucor, Torulopsis, Rhizopus, Absidia, and causative agents of coccidiodomycosis and paracoccidioidomycosis, among others.

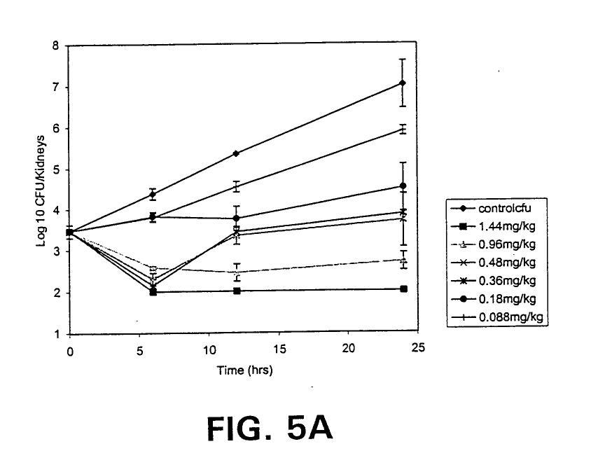

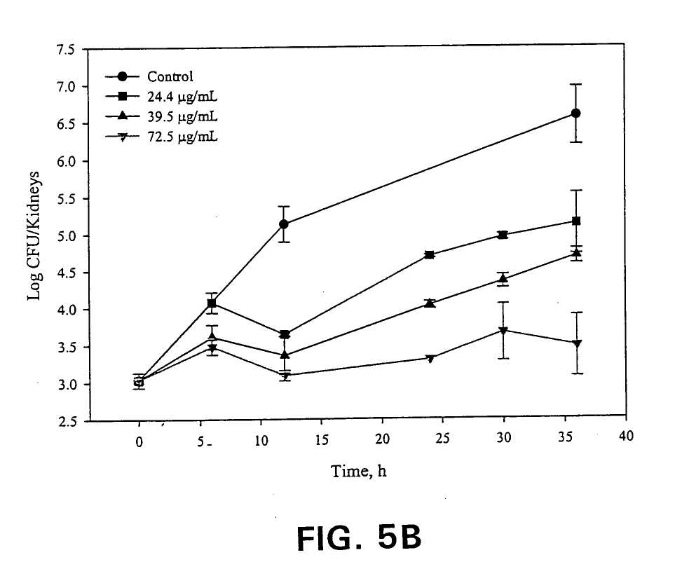

The AmB micellar formulation of the present invention was tested in a neutropenic mouse model of disseminated candidiasis. Two hours after inoculation of the mice with 105 viable cells each of Candida albicans, mice were injected intravenously with 22, 45, 90, 120 or 310 μg/ml AmB as AmB-loaded mPEG-DSPE micelles (ratio of AmB:mPEG-DSPE 0.94) or 241 μg/ml AmB as AmB-loaded mPEG-DSPE micelles (AmB:mPEG-DSPE 1.41:1). The numbers of viable C. albicans cells in the kidneys were determined after sacrifice of mice at the start of the experiment and 6, 12 and 24 hours after intravenous injection of the AmB formulations. The results are given in FIGS. 5A-5B. At the two highest doses tested there was a significant drop in viable count in the kidneys, while doses greater than about 0.1 mg/kg appeared to inhibit C. albicans growth over the time monitored. Thus, the AmB-mPEG micelles of the present invention are effective in the treatment of disseminated candidiasis in an in vivo setting. For a comparison to a commercial, FDA-approved formulation (Fungizone, AmB solubilized using sodium deoxycholate, Bristol-Myers Squibb, Princeton, N.J.), the same experiment was carried out, and monitoring was extended to 36 hours after intravenous administration of AmB. The results are shown in FIG. 5. The AmB-mPEG micellar formulation of the present invention appears to be similar in efficacy to that of the commercially available, detergent-solubilized form with respect to its in vivo microbicidal activity against C. albicans.

The direct dissolution of AmB as Fungizone® (Bristol-Myers Squibb, Princeton, N.J.) at a 2:1 molar ratio of sodium deoxycholate to AmB in water produces some monomer and various species of soluble aggregates of the drug combined with sodium deoxycholate. Similarly, AmB dissolved in dimethylformamide and diluted with excess water, produces some soluble monomer and various species of soluble aggregates. The proportion of these species of AmB depends on the method of dissolution, temperature, and concentration. In both cases, AmB will precipitate given sufficient time. In both cases, the absorption spectrum of AmB is characterized by a broad band at 328 nm, corresponding to aggregated species of AmB (A form). In contrast, AmB is entirely monomeric in dimethylformamide or methanol, and in these solvents it has a characteristic spectrum with four well-separated bands at 348, 365, 385, and 409 nm (B form). Attempts to directly dissolve AmB and mPEG-DSPE in water and obtain complete dissolution of drug were not successful. Instead, AmB and mPEG-DSPE were dissolved in methanol, the solvent was removed by rotoevaporation to make a solid film of drug and polymer, and water was added and incubated at 40° C., although 25-80° C. can be used. As a result, mPEG-DSPE micelles encapsulate AmB (Table 1). The yield of AmB (level of encapsulated drug/initial level of drug) ranges from 57 top 93%, increasing with the level of mPEG-DSPE until about 90%. The ratio of mPEG-DSPE to AmB ranges from 0.90 to 3.2 mol:mol. The level of AmB reaches 240 μg/ml after reconstitution in water, a level that permits an adequate dose for the treatment of systemic fungal diseases. Further improvement was achieved using 75° C. as the temperature for hydrating the dried film.

TABLE 1
Encapsulation of AmB by mPEG-DSPE micelles
(hydration at 40° C.)

| mPEG- | | | mPEG- | | |
|---|---|---|---|---|---|
| DSPE | AmB | Encapsulated | DSPE:AmB | | |
| (mg) | (μg) | AmB (μg) | (mol:mol) | Yield (%) | I/IV ratio |
| 2.21 | 650 | 370 | 0.90 | 57 | 2.97 |
| 3.33 | 650 | 520 | 1.0 | 80 | 1.68 |
| 4.00 | 650 | 580 | 1.1 | 90 | 1.30 |
| 5.25 | 650 | 580 | 1.4 | 90 | 0.75 |
| 7.83 | 650 | 600 | 2.0 | 93 | 0.50 |
| 11.25 | 650 | 550 | 3.2 | 85 | 0.34 |

The self-aggregation state of AmB encapsulated in mPEG-DSPE micelles varies with the ratio of mPEG-DSPE to AmB from 0.90 to 3.2 (FIG. 3). A broad band at 328 nm that is characteristic of aggregated species of AmB is predominant at 0.90. The intensity of the band at 328 nm decreases relative to the other bands at higher wavelengths that are associated with monomeric drug at 1.0. With an increase in mPEG-DSPE content, sharp bands at 368, 388, and 417 nm increase in intensity and are predominant. The ratio of the intensity at 328 nm to 417 nm, i.e. I/IV ratio, a measure of the degree of aggregation [Gruda, I. et al. (1988) Cell Biol. 66:177], varies from 2.97 to 0.34 with an increase in mPEG-DSPE content.

Thus, mPEG-DSPE micelles encapsulate AmB in a monomeric state. Deaggregation of AmB in the cores of mPEG-DSPE micelles likely occurs by interaction with distearoyl chains. A slight bathochromic shift and a difference in the intensity of bands associated with encapsulated AmB relative to the drug in methanol is an indication of this interaction in the cores. In particular, the intensity of the band at 417 nm is less than the intensity of bands at 368 and 388 nm. For AmB in methanol, it is opposite, and bands II, III and IV are at 365, 385 and 409 nm.

It was determined that hydrating the thin film of mPEG-DSPE and AmB at elevated temperature improves the yield of drug-loaded micelles. The temperature for hydrating is desirably near, desirably at or above the melting temperature of the phospholipid component of the amphiphilic polymer. Generally, the temperature can be from about 25° C. to about 80° C. For mPEG-DSPE, 75° C. provides good results. The suspension of micelles is clearer than when prepared at about 25° C., and there is less material retained on membrane after filtration of the micelle suspension. Without wishing to be bound by any particular theory, the inventor believes that the increase in phospholipid fluidity is responsible for the improved results in micellization and/or loading of micelles. It is understood that the temperature cannot be at or above either the temperature at which the amphiphilic polymer or the passenger compound decompose or lose activity or it cannot be at or above the boiling temperature of water.

Several strategies have been proposed to deaggregate and thus lower the toxicity of polyene antibiotics [Brajtburg, J. et al. (1996) J. Clinical Microbiological Reviews 9:512. While nontoxic Pluronics® (trademark, BASF, Mount Olive, N.J., polyoxyalkylene ether) were unable to deaggregate AmB, they were able to deaggregate nystatin, a structurally related polyene antibiotic, and thus reduce its toxicity in terms of hemolysis [Yu, B. et al. (2000) in Biomaterials and Drug Delivery toward a New Millennium, Park, K. D.; Kwon, I. C.; Yui, N.; Jeong, S. Y.; Park, K. Eds.] Gruda and coworkers added excess surfactant such as sodium deoxycholate or lauryl sucrose to AmB and obtained a reduction in self-aggregation and thus a reduction in acute toxicity in mice [Barwicz, J. et al. (1992) Antimicrob. Agents Chemother. 36:2310. Tasset and coworkers added excess Myrj 59, a mPEG derivative of stearic acid, (Myrj, trademark of ICI Americas Inc., Wilmington, Del.) to AmB and obtained a reduction in self-aggregation [Tasset, C. et al. (1990) J. Pharm. Pharmacol. 43:297]. In both these cases, however, the high levels of surfactant (mol ratio>50) are too toxic for use in humans. Much less mPEG-DSPE with a distearoyl chain is required than Myrj 59 with a single stearoyl chain for the deaggregation of AmB. Similarly, mPEG-block-poly(L-aspartate) with 17 stearoyl side chains also forms micelles that readily deaggregate AmB, reduces its toxicity as determined by hemolysis, but exerts no untoward hemolysis by itself [Lavasanifar, A. et al. (2002) Pharm. Res. 19:418; Adams, M. L. et al. (2003) J. Controlled Release 87:23]. AmB encapsulated in these polymeric micelles has potent in vivo antifungal activity. mPEG-DSPE has been used safely in humans with intravenous injection.

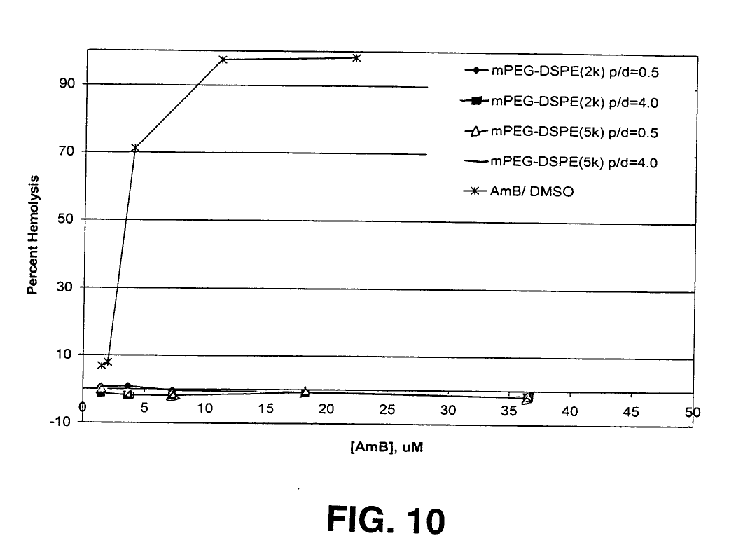

The interactions of AmB with itself, membrane sterols, and carriers are complex, and the aggregation state of the drug is a good indicator of toxicity and hemolytic activity. Consequently, the ability to modulate the equilibrium between the different aggregates is of primary concern for AmB formulation development. The incorporation of AmB in micelles with mPEG-PL, especially mPEG-DSPE, has a profound influence on the aggregation state of the encapsulated AmB. In turn, the relative aggregation state affects the hemolytic activity of AmB toward mouse erythrocytes. A comparison of the hemolytic activity of AmB with AmB incorporated within micelles with mPEG(2k)-DSPE, mPEG(5k)-DSPE is shown in FIG. 10. mPEG-DSPE was used at ratios of polymer to drug of 0.5 and 4.0 for both lengths of the mPEG polymer in the DSPE complex. Over the range of concentrations tested, neither the mPEG chain length nor the ratio appeared to affect the results. Thus, the micelles effectively prevented aggregation of the AmB, as measured by the hemolysis of mouse red cells.

Anticancer agents such as adriamycin, paclitaxel, taxol and comptothecin are also reduced in toxicity (and improved with respect to water solubility) when encapsulated in micelles according to the present invention and delivered by parenteral administration, for example by intravenous injection or infusion.

It is preferred that the drug-loaded micelles of the present invention are freeze-dried after preparation and stored in the dry state in a manner consistent with maintenance of the activity of the drug, as known in the art for a particular drug. The dry micelles are reconstituted in a pharmaceutically acceptable carrier such as sterile physiological saline or a sterile dextrose solution, e.g., 5% dextrose, and after thorough hydration, they can be filtered (optionally through a 0.22 μm filter) prior to administration. Alternatively, a sugar (e.g., trehalose, sucrose, mannitol, among others) can be incorporated prior to freeze-drying in an amount sufficient to improve the ease of reconstitution and/or to improve the stability of the micelles when reconstituted prior to use in the treatment of a patient. The therapeutic AmB micelles of the present invention are administered parenterally at dosages from about 0.1 to about 5 mg/kg/day.

All references cited in the present application are incorporated in their entirety herein by reference to the extent not inconsistent herewith.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1

AmB Encapsulation by mPEG-DSPE Micelles

AmB was obtained from Chem-lmpex (Wood Dale, Ill.). mPEG-DSPE (MPEG: Mₙ=5000 g/mol) was purchased from Nektar Therapeutics (Birmingham, Ala.).

AmB (654 μg) and mPEG-DSPE (2.21, 3.33, 4.00, 5.25, 7.83 or 11.25 mg) were dissolved in 5.0 ml methanol in a vial. The contents of the vial were sonicated (Fisher Ultrasonic Waterbath) for 5 min until a clear solution was produced. The clear solution was then transferred to a round bottom flask. The solvent was evaporated at 40° C. under reduced pressure (300 mm Hg) using a rotoevaporator to form a thin film of mPEG-DSPE and AmB.

The thin film was dissolved in 5.0 ml distilled water in the round bottom flask by incubating at 40° C. for 10 min and then vortexing the contents of the flask for 30 sec. The micellar solution was filtered using a 0.22 μm membrane. Aliquots of 0.5 ml were transferred to vials and lyophilized overnight. Table 1 shows the results.

### Example 2

Spectrophotometric Analysis of AmB in mPEG-DSPE Micelles

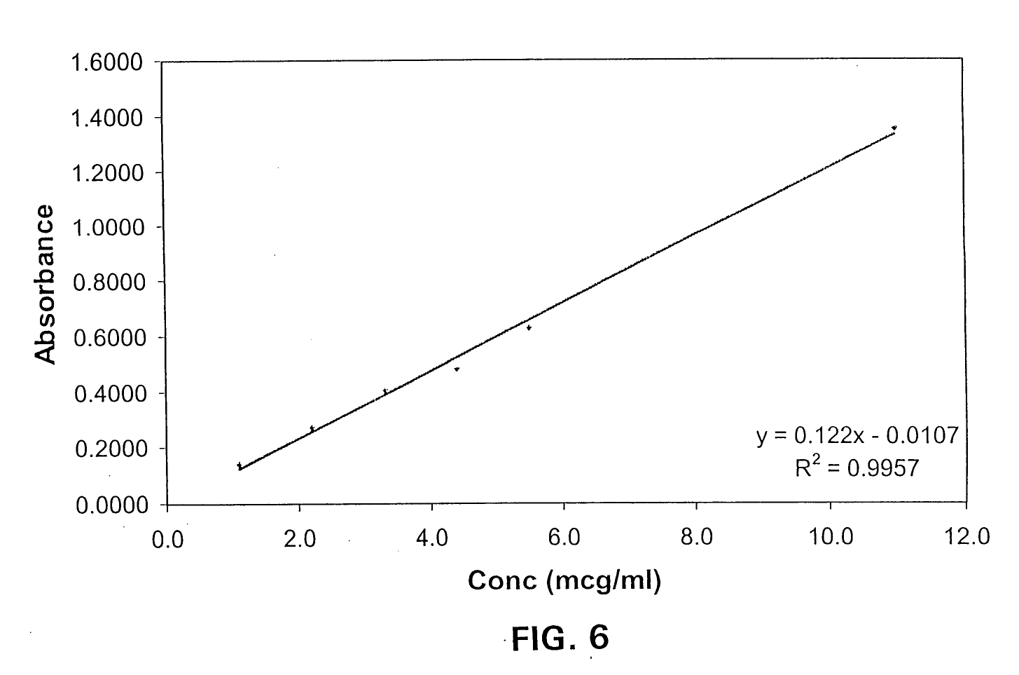

A standard curve of AmB in DMF:water (1:1) (filtered) was derived as follows. Concentrations of AmB in DMF:water were prepared (1.1, 2.2, 3.3, 4.4, 5.5 and 11 μg/ml). Absorbances of these solutions were determined spectrophotometrically (412 nm), and absorbance was plotted against concentration. See FIG. 6. The standard curve was used to calculate the percent yield of AmB encapsulated in micelles.

Prior to analysis, 4.5 ml distilled water was added to a vial containing the lyophilized micelles, and the contents were vortexed for 30 sec. Aliquots were prepared with DMF:water (1:1). Then 1.5 ml of the solution was transferred to a cuvette. The amount of AmB encapsulated was determined spectrophotometrically at 412 nm, with reference to the standard curve.

To produce spectra for determining aggregation of AmB, freeze-dried samples of AmB/mPEG-DSPE micelles were reconstituted using distilled water and filtered, and absorbances were determined at 328 and 412 nm, and the ratio of the absorbance at 328 to that at 412 nm was determined. A UV-VIS spectrum was produced and recorded using a scan range of 300-450 nm and a scan step of 0.1 nm.

### Example 3

In Vivo Antifungal Activity

The neutropenic mouse model of candidiasis was used to test the efficacy of AmB formulations (Andes et al. (2001) Antimicrob. Agents Chemother. 45:922-926). Candida albicans strain K-1 (a clinical bloodstream isolate) was maintained, grown, subcultured and quantified on Sabaroud dextrose agar (Difco Laboratories, Detroit, Mich.). Fungizone was from Bristol-Myers Squibb (Princeton, N.J.). 24 hrs prior to the start of an experiment, the organisms were subcultured at 35° C. In a first experiment, AmB loaded in mPEG-DSPE micelles was diluted in water to contain 2120 μg/ml mPEG-DSPE and 241 μg AmB (theoretical molar ratio of polymer:drug is 1.41:1). A second experiment used 2120 μg polymer and 361 μg AmB (theoretical molar ratio of polymer:drug is 0.94:1).

The body weights of six week-old specific-pathogen-free female ICR/Swiss mice (Harlan Sprague Dawley, Madison, Wis.) were between 23 and 27 g, with an average weight of 25 g. Mice were rendered neutropenic (<100 polymorphonuclear leukocytes) by intraperitoneal injection with cyclophosphamide (Mead Johnson Pharmaceuticals, Evansville, Ill.) (150 mg/kg body weight) 4 days and 1 day (100 mg/kg body weight) before infection.

The AmB-mPEG-DSPE micelles were dissolved in Sterile Water of Injection, and a constant volume of 0.1 ml was given intravenously in all cases. The various concentrations studied were 361, 120, 90, 45 and 22 μg/ml (theoretical molar ratio of polymer:drug of 1.41:1) and 241 μg/ml (theoretical molar ratio of polymer: drug of 1.41:1).

C. albicans was subcultured 24 hrs before infection of mice. Cells from 6 colonies were suspended in sterile pyrogen-free 0.9% saline warmed to 35° C. Fungal counts of the inoculum were determined on SDA to be 10⁶ CFU/ml.

Disseminated infection with the pathogenic C. albicans was achieved by injecting 0.1 ml of inoculum (10⁵ CFU injected) via the lateral tail vein 2 hr prior to the start of drug therapy. At the end of the therapy period, the mice were sacrificed by CO₂ asphyxiation. After sacrifice the kidneys of each mouse were immediately removed and placed in sterile 0.9% saline at 4° C. The homogenized mixture was serially diluted and aliquots were plated on SDA for colony counts (24 hr incubation at 35° C.). The lower limit of detection was 100 CFU/ml. Results were expressed as the mean CFU/per kidney for two mice. See Table 2 and FIGS. 5A-5B.

Regression polynomial for the dependent variable, ratio of AmB peak I and peak IV (ration 328/412) was calculated and applied to approximate the response surface and contour plot. The final model for I/IV ratio is as follows:

I/IV ratio=6.825−0.00913x−0.02y+0.00004507xy−0.0000449x²+0.00001741y²−0.00000000062x²y²−0.0000000000848x⁴−0.00000000000473y⁴+0.000000002008x³y+0.00000000003556xy³

R²=0.963(F=113.889 p<0.01)

where x=concentration of AmB (0.5 mg/ml) and y=concentration of mPEG-DSPE (0.5 mg/ml).

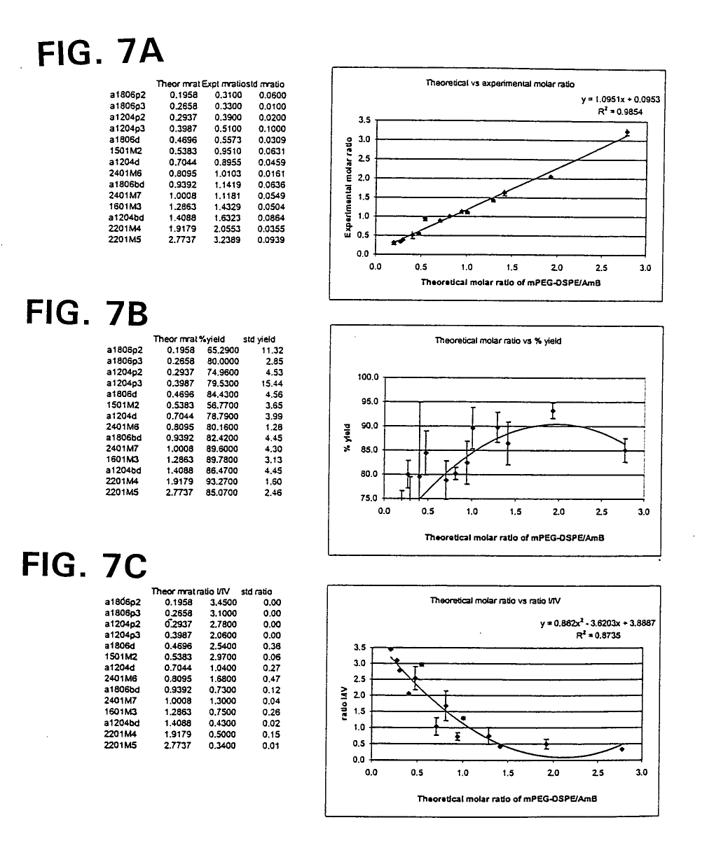

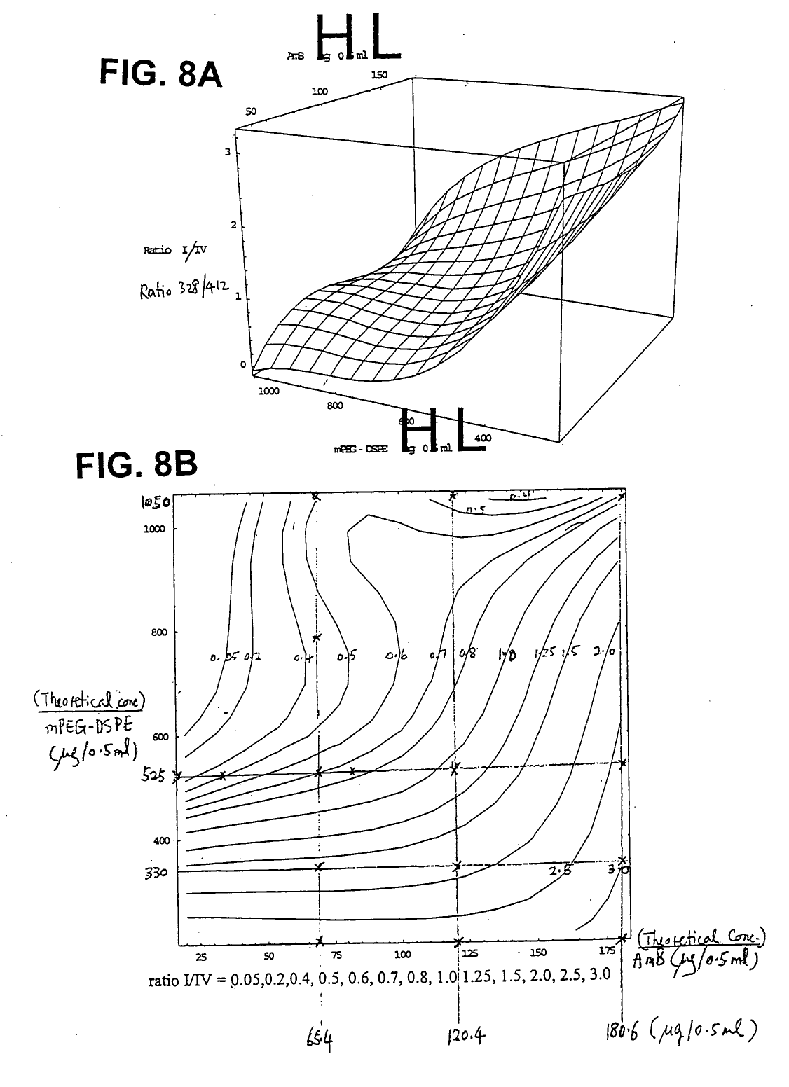

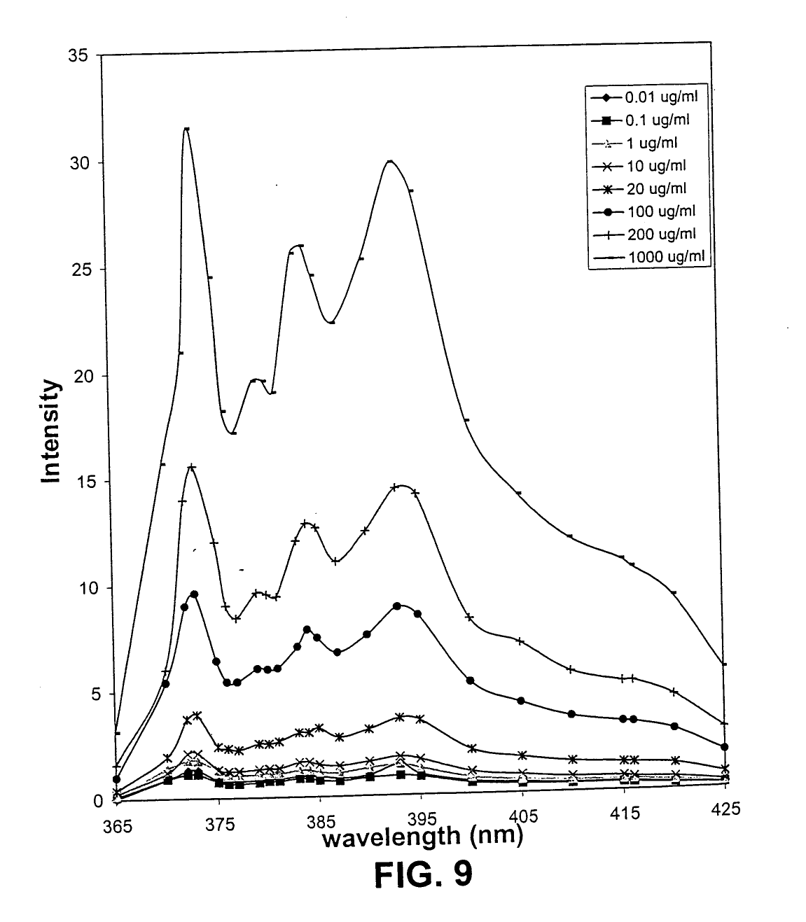

Data are presented in Table 2 and FIGS. 7A-7C and 8A-8B.

### Example 4

Assessment of Hemolytic Activity of AmB/Polymer Formulations.

Murine blood was collected by cardiac puncture; heparin was used as an anticoagulant. The erythrocytes were separated by centrifugation and washed using isotonic PBS. The cell pellet was diluted appropriately in PBS to obtain suspensions of 5×10⁷ cells/ml.

The AmB/polymer formulations and polymer blanks were brought to room temperature and reconstituted with 1.0 ml of PBS just prior to use. 1 ml of the red cell suspension was incubated with 1 ml AmB preparation (36, 18, 7, 4, 1 μM)at 37° C. for 30 min. at 37° C. The cells were then centrifuged at 3000 rpm and the absorbance of the supernatant was measured at 542.

Complete lysis of the red cells was obtained by diluting the stock red cell solution in water, causing osmotic rupture. The absorbance at 542 nm for samples with complete hemolysis is recorded as A100. The percent hemolysis for the AmB samples was reported as (A-A0/A100-A0]*100, where A0 is the absorbance for control samples (buffer only).

As a control, the hemolysis experiment was performed using the aggregated form of the drug. A stock solution of AmB (43 μM) was prepared by dissolving 4 mg AmB in 0.5 ml DMSO and then diluting with isotonic PBS to 100 ml. This stock solution was diluted with PBS to obtain lower concentrations and the experiment was performed at 22, 11, 4, 2 and 1.5 μM AmB. Concentrations of AmB formulated in mPEG-DSPE micelles were tested; see also FIG. 10 and its description.

A solution containing 8 mg/ml of amphotericin B in dimethyl sulfoxide (DMSO) was prepared and diluted with buffer to give 6 μg/ml AmB in PBS containing 0.075% DMSO. 1.0 ml of the 1.48×10⁸ cells/ml suspension was incubated with 1.0 ml of 6 μg/ml AmB in PBS (0.075% DMSO) or PBS at 37° C. in the shaking water bath (75 rpm) for 24 h. In all cases, the final AmB concentration in the incubated samples was approximately 3 μg/ml. The final cell concentration was 7.2×10⁷ erythrocytes/ml. Samples, blanks, and controls were withdrawn in triplicate at 1, 9, 16, and 24 h and centrifuged at 3000 rpm for 10 min. The supernatant was collected, and hemoglobin content was determined by absorbance at 542 nm. The values for total cell lysis were obtained by hypotonic hemolysis. Percent hemolysis is reported by 100(Absₛ−Abs_{b})/(Abs₁−Abs_{b}) where Absₛ is the absorbance of the sample, Abs_{b} is the average absorbance of the buffer, and Abs₁ is the average absorbance of the lysed samples. All values are reported as mean±standard deviation.

TABLE 2
Amphotericin B loaded mPEG-DSPE micelles and in vivo time kill in a neutropenic murine model of disseminated candidiasis.

| | | 361 μg/ml | 241 μg/ml | | | | | IV injection |
|---|---|---|---|---|---|---|---|---|
| Time | control | 1.44 | 0.96 | 120 μg/ml | 90 μg/m | 45 μg/ml | 22 μg/ml | Volume given = 0.1 ml |
| (h) | cfu | mg/kg | mg/kg | 0.48 mg/kg | 0.36 mg/kg | 0.18 mg/kg | 0.088 mg/kg | Mouse body Wt = 23-27 g 1.444 |
| 0 | 3.47 | 3.47 | 3.47 | 3.47 | 3.47 | 3.47 | 3.47 | 0.964 |
| 6 | 4.38 | 2 | 2.58 | 2.3 | 2.15 | 3.82 | 3.8 | 0.48 |
| 12 | 5.35 | 2 | 2.45 | 3.35 | 3.44 | 3.76 | 4.54 | 0.36 |
| 24 | 7.01 | 2 | 2.71 | 3.71 | 3.87 | 4.51 | 5.89 | 0.18 |
| | | | | | | | | 0.088 |
| | control sd | sd1 | sd2 | sd3 | sd4 | sd5 | sd6 | |
| | 0.16 | 0.16 | 0.16 | 0.16 | 0.16 | 0.16 | 0.16 | |
| | 0.14 | 0 | 0.03 | 0.15 | 0.15 | 0.11 | 0.11 | |
| | 0.02 | 0 | 0.21 | 0.22 | 0.1 | 0.3 | 0.13 | |
| | 0.57 | 0 | 0.21 | 0.65 | 0.05 | 0.57 | 0.11 | |

What is claimed is:

1. A method for formulating a deaggregated polyene antibiotic, said method comprising the steps of:
(a) dissolving a polyene antibiotic and a poly(ethylene glycol)-phospholipid in a solvent to produce a solution;
(b) evaporating the solvent from the solution of step (a) under conditions of temperature from 26° C. to 40° C. and conditions of pressure from 100 mm to 300 mm mercury to produce a drug-polymer film;
(c) adding water at a temperature from 25° C. to 80° C. to the drug-polymer film of step (b) and mixing vigorously,
whereby micelles comprising the polyene antibiotic and poly(ethylene glycol)-phospholipid are formed.

2. The method of claim 1 wherein the poly(ethylene glycol)-phospholipid is monomethoxy poly(ethylene glycol)-1,2-di-stearoyl-phosphatidyl ethanolamine.

3. The method of claim 1 wherein the polyene antibiotic is Amphotericin B (AmB).

4. The method of claim 1 wherein the solvent is methanol or chloroform: methanol (1:2).

5. The method of claim 4 wherein the conditions for evaporating the solvent are 40° C. and 300 mm mercury.

6. The method of claim 3 wherein in step (c) water is added at a temperature from 40° C. to 75° C.

7. The method of claim 2 wherein the molecular weight of the poly(ethylene glycol)-1,2-di-stearoyl-phosphatidyl ethanolamine is about 5000 to about 12,000.

8. The method of claim 3 wherein the poly(ethylene glycol)-phospholipid is monomethoxy poly(ethylene glycol)-1,2-di-stearoyl-phosphatidyl ethanolamine and the molar ratio of AmB to poly(ethylene glycol)-1,2-di-stearoyl-phosphatidyl ethanolamine to AmB is from about 0.75:1 to about 10:1.

9. The method of claim 8 wherein the molar ratio of poly(ethylene glycol)-1,2-di-stearoyl-phosphatidyl ethanolamine to AmB is from 1:1 to 3:1.

10. The method of claim 9 wherein the molar ratio of poly(ethylene glycol)-1,2-di-stearoyl-phosphatidyl ethanolamine to AmB is 1:1 to 1.5:1.

11. The method of claim 1 further comprising the step of freeze-drying the micelles after step (c).

12. A composition comprising micelles consisting essentially of Amphotericin B (AmB) and poly(ethylene glycol)-1,2-di-stearoyl-phosphatidyl ethanolamine (mPEG-DSPE) in a molar ratio of mPEG-DSPE:AmB of from 1:1 to 3:1.

13. The composition of claim 12 further comprising a pharmaceutically acceptable carrier.

14. The composition of claim 13 wherein the pharmaceutically acceptable carrier is a dextrose solution.
